(19) United States
(12) Patent Application Publication
(10) Pub. No.: US 2022/0361772 A1
(43) Pub. Date: Nov. 17, 2022

(54) GAIT STATE MEASUREMENT SYSTEM, GAIT STATE MEASUREMENT METHOD, AND PROGRAM

(71) Applicant: TOYOTA JIDOSHA KABUSHIKI KAISHA, Toyota-shi (JP)

(72) Inventors: Yoshinori HAYAKAWA, Owariasahi-shi (JP); Taiga MATSUMOTO, Nagoya-shi (JP)

(21) Appl. No.: 17/659,944

(22) Filed: Apr. 20, 2022

(30) Foreign Application Priority Data
May 12, 2021 (JP) 2021-081136

(51) Int. Cl. A61B 5/11; A61B 5/103; A61B 5/00; G06T 7/70

(52) U.S. Cl. CPC A61B 5/112; A61B 5/1036; A61B 5/74; G06T 7/70; A61B 2562/0252; G06T 2207/30196

(57) ABSTRACT

A gait state measurement device measures a gait state of a subject who is walking on a walking surface formed on a belt that runs along a circulation track. The gait state measurement device includes an acquisition unit, a position estimation unit, and a warning control unit. The acquisition unit acquires measurement information of a load distribution sensor that detects the load of the subject through the belt. The position estimation unit estimates the position of the sole of the subject based on the measurement information. The warning control unit performs a different type of warning depending on a relationship between the position of the sole of the subject and each of target areas that are defined according to an installation area of the load distribution sensor.

### CROSS-REFERENCE TO RELATED APPLICATION

This application claims priority to Japanese Patent Application No. 2021-081136 filed on May 12, 2021, incorporated herein by reference in its entirety.

### BACKGROUND

### 1. Technical Field

The disclosure relates to a gait state measurement system, a gait state measurement method, and a program.

### 2. Description of Related Art

A gait training system for a patient undergoing rehabilitation (hereinafter referred to as rehab) to receive training in walking motion is being developed. In the gait training system, a load distribution of a trainee is measured by a load distribution sensor installed in a treadmill. For example, WO 2006/106714 discloses a pressure distribution detection device including two types of loop electrode groups, an elastic body placed on the loop electrode groups, and conductive members placed on the elastic body. The gait training system measures a gait state of the trainee based on a measurement result and assists the trainee in moving his or her joint.

### SUMMARY

The load distribution sensor is installed in a treadmill main body under a moving belt, independently of the belt. Therefore, the relationship between the position of the sole of the trainee and the position of a sensor area cannot be recognized from the outside. This poses a problem in that, when the sole of the trainee has deviated from a proper position during gait training, such as when the sole has deviated or is about to deviate from the sensor area, the trainee or a therapist or the like cannot know it.

Having been contrived to solve this problem, the disclosure provides a gait state measurement system, a gait state measurement method, and a program that appropriately notify that the sole of a subject has deviated from a proper position during gait measurement.

A gait state measurement system according to one aspect of the disclosure is a gait state measurement system that measures a gait state of a subject who is walking on a walking surface formed on a belt running along a circulation track. The gait state measurement system includes an acquisition unit, a position estimation unit, and a warning control unit. The acquisition unit acquires measurement information of a load distribution sensor that detects the load of the subject through the belt. The position estimation unit estimates the position of the sole of the subject based on the measurement information. The warning control unit performs a different type of warning depending on a relationship between the position of the sole of the subject and each of target areas that are defined according to an installation area of the load distribution sensor. Thus, the subject or an assistant can be appropriately notified of to what extent a treading position of the subject is at a proper position and of deviation from the proper position.

Here, the target areas may include a first target area that is located inside the installation area and a second target area that encompasses the first target area. The warning control unit may perform a first warning when at least part of the sole of the subject deviates from the first target area while an entire sole of the subject is located inside the second target area. The warning control unit may perform a second warning when at least part of the sole of the subject deviates from the second target area. By thus giving warnings in stages, the gait state measurement system can more clearly notify the subject or the assistant of to what extent the treading position is at the proper position.

The position estimation unit may estimate the position of the sole of the subject based on a captured image created by imaging the subject in walking and on the measurement information. The warning control unit may perform a third warning when the entire sole of the subject deviates from the second target area. Thus, the gait state measurement system can notify the subject or the assistant that the treading position has deviated significantly. Therefore, the subject or the assistant can be quickly prompted to make a decision, such as suspending the gait measurement or the gait training.

The warning control unit may perform a fourth warning when the entire sole of the subject is not located inside the first target area within a predetermined time from the first warning. Thus, the subject or the assistant can be notified that the treading position has not improved and be appropriately prompted to improve it.

The gait state measurement system may include a target area alteration unit that alters at least one of the position, the area, and the shape of the target area based on at least one of attribute information on the subject and an initial position of the sole of the subject. Thus, the gait state measurement system has enhanced convenience and can perform smooth measurement.

The gait state measurement system may include a record unit that records a history of the warnings in a warning log. The gait state measurement system may include a target area alteration unit that alters at least one of the position, the area, and the shape of the target area based on at least either the number of times of warnings or a chronological pattern of warnings recorded in the warning log. Thus, the convenience can be enhanced according to the current actual situation of measurement.

A gait training system according to one aspect of the disclosure includes: a belt that runs along a circulation track and forms a walking surface on which a subject walks; a load distribution sensor that detects the load of the subject through the belt; and a gait state measurement device that measures a gait state of the subject. The gait state measurement device includes an acquisition unit, a position estimation unit, and a warning control unit. The acquisition unit acquires measurement information of the load distribution sensor. The position estimation unit estimates the position of the sole of the subject based on the measurement information. The warning control unit performs a different type of warning depending on a relationship between the position of the sole of the subject and each of target areas that are defined according to an installation area of the load distribution sensor. Thus, the subject or the assistant can be appropriately notified of to what extent a treading position of the subject is at a proper position and of deviation from the proper position.

A gait state measurement method according to one aspect of the disclosure is a gait state measurement method that measures a gait state of a subject who is walking on a walking surface formed on a belt that runs along a circulation track. The gait state measurement method includes: an acquisition stage of acquiring measurement information of a load distribution sensor that detects the load of the subject through the belt; a position estimation stage of estimating the position of the sole of the subject based on the measurement information; and a warning control stage of performing a different type of warning depending on a relationship between the position of the sole of the subject and each of target areas that are defined according to an installation area of the load distribution sensor. Thus, the subject or the assistant can be appropriately notified of to what extent a treading position of the subject is at a proper position and of deviation from the proper position.

A program according to one aspect of the disclosure is a program that causes a computer to execute a gait state measurement method that measures a gait state of a subject who is walking on a walking surface formed on a belt that runs along a circulation track. The gait state measurement method includes: an acquisition stage of acquiring measurement information of a load distribution sensor that detects the load of the subject through the belt; a position estimation stage of estimating the position of the sole of the subject based on the measurement information; and a warning control stage of performing a different type of warning depending on a relationship between the position of the sole of the subject and each of target areas that are defined according to an installation area of the load distribution sensor. Thus, the subject or the assistant can be appropriately notified of to what extent a treading position of the subject is at a proper position and of deviation from the proper position.

The disclosure can provide a gait state measurement system, a gait state measurement method, and a program that appropriately notify that the sole of a subject has deviated from a proper position during gait measurement.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a schematic perspective view of a gait training system according to Embodiment 1;

FIG. 2 is a schematic perspective view showing one example of the configuration of a gait assistive device;

FIG. 3 is a side view and a top view of a treadmill according to Embodiment 1;

FIG. 4 is a block diagram showing the schematic configuration of a gait state measurement device according to Embodiment 1;

FIG. 5 is a view illustrating types of warnings according to Embodiment 1;

FIG. 6 is a view showing one example of a display of a training monitor according to Embodiment 1;

FIG. 7 is a flowchart showing the procedure of a process executed by the gait state measurement device according to Embodiment 1;

FIG. 8 is a view illustrating a process of predicting the position of a sole area according to Embodiment 1;

FIG. 9 is a block diagram showing the schematic configuration of a gait state measurement device according to Embodiment 2;

FIG. 10 is a view showing one example of target areas according to Embodiment 2;

FIG. 11 is a view showing one example of the target areas according to Embodiment 2;

FIG. 12 is a flowchart showing the procedure of a process executed by the gait state measurement device according to Embodiment 2;

FIG. 13 is a block diagram showing the schematic configuration of a gait state measurement device according to Embodiment 3;

FIG. 14 is a view showing one example of the data structure of a warning log according to Embodiment 3;

FIG. 15 is a view showing one example of the data structure of a control table according to Embodiment 3; and

FIG. 16 is a schematic configuration diagram of a computer used as the gait state measurement devices according to Embodiments 1 to 3.

### DETAILED DESCRIPTION OF EMBODIMENTS

The disclosure will be described below through embodiments, but it is not intended to limit the disclosure according to the claims to the following embodiments. Not all components to be described in the embodiments are necessarily essential as solutions to the problem. To clarify the description, the following text and the drawings are abbreviated and simplified as necessary.

### Embodiment 1

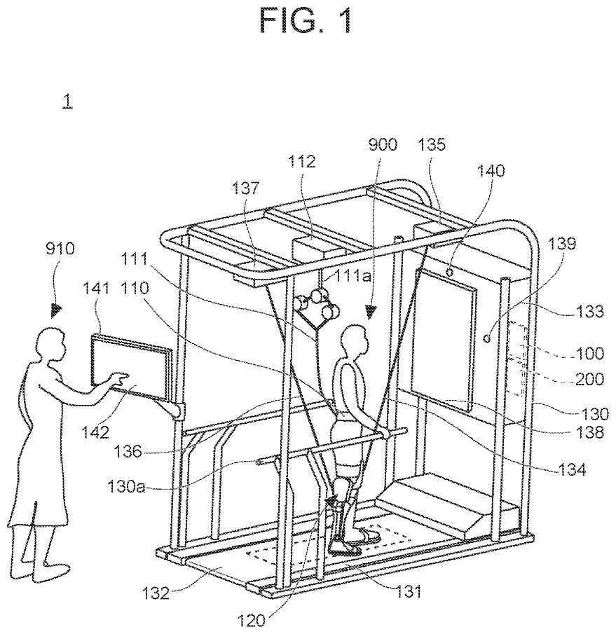

First, Embodiment 1 of the disclosure will be described. FIG. 1 is a schematic perspective view of a gait training system 1 according to Embodiment 1. The gait training system 1 is one example of a system to which a gait state measurement device (also called a gait state measurement system) according to Embodiment 1 can be applied. The gait training system 1 is a system for a trainee 900 who is a hemiplegia patient suffering from paralysis in one of his or her legs to receive gait training. The trainee 900 is also called a subject. An up-down direction, a left-right direction, and a front-rear direction in the following description are directions based on the directions of the trainee 900.

The gait training system 1 mainly includes a control panel 133 that is mounted on a frame 130 constituting an overall framework, a treadmill 131 on which the trainee 900 walks, and a gait assistive device 120 that is worn on an affected leg that is the leg on the paralyzed side of the trainee 900.

The frame 130 is erected on the treadmill 131 that is installed on a floor. The treadmill 131 rotates a ring-shaped belt 132 by a motor (not shown). Thus, the belt 132 runs along a circulation track. The treadmill 131 is a device that prompts the trainee 900 to walk. The trainee 900 receiving gait training rides the belt 132 and tries to perform walking motion on a walking surface formed on the belt 132.

The frame 130 supports the control panel 133, a training monitor 138, and a voice output unit 139.

The control panel 133 houses a system control unit 200 and a gait state measurement device 100. The system control unit 200 is a computer device that controls motors and sensors. The gait state measurement device 100 is a computer device that measures a gait state of the trainee 900 who is walking on the walking surface from measurement results of sensors.

The training monitor 138 is a display device that presents the trainee 900 with information about training and measurement. The training monitor 138 is, for example, a liquid crystal panel. The training monitor 138 is installed in such a manner that the trainee 900 can view it while walking on the belt 132 of the treadmill 131.

The voice output unit 139 outputs information about training and measurement by voice to notify the trainee 900. The voice output unit 139 is, for example, a speaker. The voice output unit 139 is installed at such a position that the trainee 900 can hear the voice while walking on the belt 132 of the treadmill 131.

The frame 130 supports a front-side tension unit 135, a harness tension unit 112, and a rear-side tension unit 137 respectively near a front side of an overhead position of the trainee 900, near the overhead position, and near a rear side of the overhead position. The frame 130 may include a handrail 130a for the trainee 900 to grasp.

A camera 140 is a front-side camera unit that images the trainee 900 at such an angle of view that the gait of the trainee 900 can be recognized from the front side. The camera 140 may include a lateral-side camera unit that images the trainee 900 at such an angle of view that the gait of the trainee 900 can be recognized from a lateral side. The camera 140 in this embodiment includes a set of a lens and an imaging device that have such an angle of view that the whole body, including the head, of the trainee 900 standing on the belt 132 can be captured. The imaging device is, for example, a CMOS image sensor and converts an optical image formed in an image formation plane into an image signal. The camera 140 is installed in the vicinity of the training monitor 138 so as to face the trainee 900. When the camera 140 includes the lateral-side camera unit, the lateral-side camera unit may be installed on the handrail 130a so as to capture the trainee 900 from the lateral side.

A front-side wire 134 is coupled at one end to a winding mechanism of the front-side tension unit 135 and at the other end to the gait assistive device 120. The winding mechanism of the front-side tension unit 135 turns a motor (not shown) on and off in accordance with a command from the system control unit 200 and thereby winds and unwinds the front-side wire 134 according to the motion of the affected leg. Similarly, a rear-side wire 136 is coupled at one end to a winding mechanism of the rear-side tension unit 137 and at the other end to the gait assistive device 120. The winding mechanism of the rear-side tension unit 137 turns a motor (not shown) on and off in accordance with a command from the system control unit 200 and thereby winds and unwinds the rear-side wire 136 according to the motion of the affected leg. As the front-side tension unit 135 and the rear-side tension unit 137 thus operate in a coordinated manner, the load of the gait assistive device 120 is canceled so as not to burden the affected leg, and further swing-forward motion of the affected leg is assisted according to a set degree.

An operator 910 who is a training assistant sets an assistance level to a higher level for a trainee who suffers from severe paralysis. The operator 910 is a physiotherapist or a doctor who has the authority to select, correct, and add setting items of the gait training system 1. When the assistance level is set to a higher level, the front-side tension unit 135 winds the front-side wire 134 with a relatively high force at a timing when the affected leg is swung forward. When the training progresses and assistance becomes unnecessary, the operator sets the assistance level to a minimum level. When the assistance level is set to the minimum level, the front-side tension unit 135 winds the front-side wire 134 with a force sufficient to cancel the weight of the gait assistive device 120 itself at a timing when the affected leg is swung forward.

The gait training system 1 includes a safety device that has safety equipment 110, a harness wire 111, and the harness tension unit 112 as main constituent elements. The safety equipment 110 is a belt that is wound around the abdomen of the trainee 900, and is fixed to the hips with, for example, a touch-and-close fastener. The harness wire 111 is a wire that is coupled at one end to the safety equipment 110 and at the other end to a winding mechanism of the harness tension unit 112. The harness wire 111 has a harness belt 111a at a portion coupled to the winding mechanism of the harness tension unit 112. The harness belt 111a is a belt that covers the harness wire 111 on the side of the portion coupled to the winding mechanism of the harness tension unit 112. The winding mechanism of the harness tension unit 112 turns a motor (not shown) on and off and thereby winds and unwinds the harness wire 111. In this configuration, when the trainee 900 significantly loses his or her balance, the safety device supports the upper body of the trainee 900 by the safety equipment 110 by winding the harness wire 111 in accordance with a command from the system control unit 200 that has detected the motion of the trainee 900.

A management monitor 141 is a display device that is mounted on the frame 130 to be monitored and operated by the operator 910. The management monitor 141 is, for example, a liquid crystal panel, and a touch panel 142 as one example of an input device is superimposed on a front surface thereof. The management monitor 141 presents various menu items relating to settings of training and measurement, various parameter values during training and measurement, measurement results during training, etc. The operator 910 selects, corrects, and adds setting items through the touch panel 142, a keyboard (not shown), or the like. The management monitor 141 is installed at such a position that the trainee 900 cannot view the display thereof from a position on the treadmill 131 where the trainee 900 undergoes training trial. A support part that supports the management monitor 141 may have a rotary mechanism that reverses the display surface to accommodate a situation where the operator 910 tries to intentionally show the display screen to the trainee 900.

The gait assistive device 120 is worn on the affected leg of the trainee 900 and assists the trainee 900 in walking by reducing the burden of stretching and bending the knee joint of the affected leg. The gait assistive device 120 sends data about movement of the legs obtained by gait training to the system control unit 200, and drives a joint part in accordance with a command from the system control unit 200. The gait assistive device 120 can also be connected through a wire or the like to a hip joint (a connection member having a rotary part) mounted on the safety equipment 110 that is a part of a fall-prevention harness device.

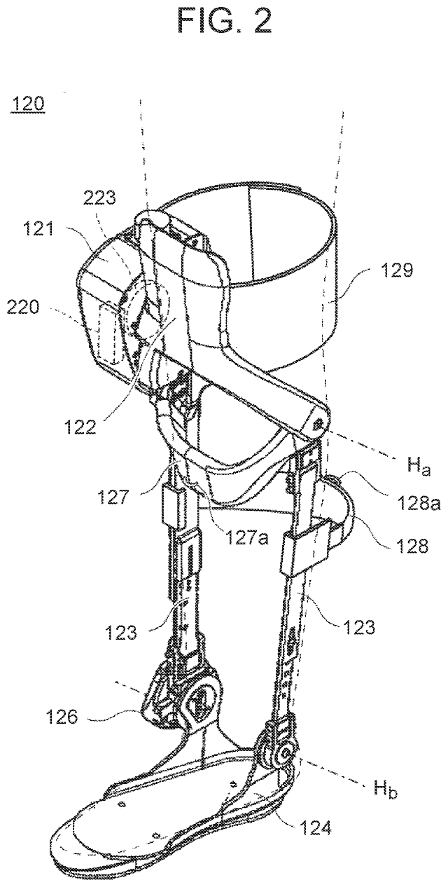

FIG. 2 is a schematic perspective view showing one example of the configuration of the gait assistive device 120. The gait assistive device 120 mainly includes a control unit 121 and a plurality of frames that supports parts of the affected leg. The gait assistive device 120 is also called a leg robot.

The control unit 121 includes an assistance control unit 220 that performs control of the gait assistive device 120, and further includes a motor (not shown) that generates drive power for assisting stretching movement and bending movement of the knee joint. The frames that support parts of the affected leg include an upper leg frame 122 and lower leg frames 123 that are turnably coupled to the upper leg frame 122. These frames further include a foot frame 124 that is turnably coupled to the lower leg frames 123, a front-side coupling frame 127 to which the front-side wire 134 is coupled, and a rear-side coupling frame 128 to which the rear-side wire 136 is coupled.

The upper leg frame 122 and the lower leg frames 123 turn relatively around a hinge shaft Ha shown in FIG. 2. The motor of the control unit 121 rotates in accordance with a command from the assistance control unit 220, and thereby urges the upper leg frame 122 and the lower leg frames 123 so as to open or close relatively around the hinge shaft Ha. An angle sensor 223 housed in the control unit 121 is, for example, a rotary encoder, and detects an angle formed between the upper leg frame 122 and the lower leg frames 123 around the hinge shaft Ha. The lower leg frames 123 and the foot frame 124 turn relatively around hinge shafts Hb shown in FIG. 2. An angular range of their relative turning is adjusted beforehand by means of an adjustment mechanism 126.

The front-side coupling frame 127 is provided so as to extend in the left-right direction on a front side of the upper leg and be connected at both ends to the upper leg frame 122. A coupling hook 127a to which the front-side wire 134 is coupled is provided near the center of the front-side coupling frame 127 in the left-right direction. The rear-side coupling frame 128 is provided so as to extend in the left-right direction on a rear side of the lower leg and be connected at both ends respectively to the lower leg frames 123 that extend in the up-down direction. A coupling hook 128a to which the rear-side wire 136 is coupled is provided near the center of the rear-side coupling frame 128 in the left-right direction.

The upper leg frame 122 includes an upper leg belt 129. The upper leg belt 129 is a belt provided integrally with the upper leg frame, and is wound around an upper leg part of the affected leg to fix the upper leg frame 122 to the upper leg part. This prevents the entire gait assistive device 120 from becoming displaced relatively to the leg of the trainee 900.

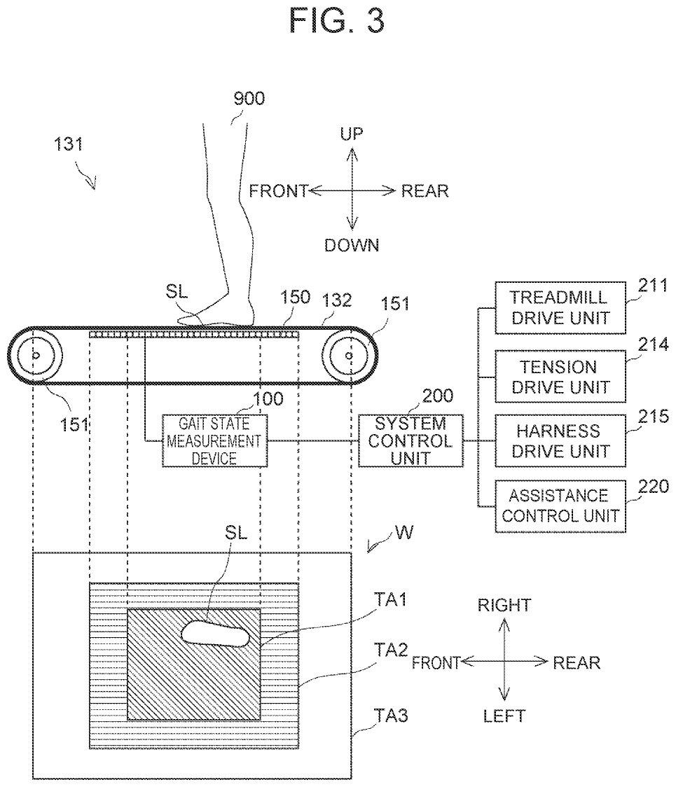

FIG. 3 is a side view and a top view of the treadmill 131 according to Embodiment 1. The treadmill 131 includes at least the ring-shaped belt 132, pulleys 151, and the motor (not shown).

The pulleys 151 are connected to the system control unit 200. The system control unit 200 rotates the belt 132 by rotating the pulleys 151.

A load distribution sensor 150 is disposed on an inside of the belt 132, i.e., a side of the belt 132 opposite from the surface on which the trainee 900 rides. The load distribution sensor 150 is fixed on a main body of the treadmill 131 without following the motion of the belt 132.

The load distribution sensor 150 is a load distribution sensor sheet having a plurality of pressure detection points. These pressure detection points are disposed in a matrix form, parallel to a walking surface W (placing surface) that supports a sole SL of the trainee 900 in a standing state. The load distribution sensor 150 is disposed on a center side of the walking surface W in the left-right direction orthogonal to a walking front-rear direction. The walking front-rear direction is a direction parallel to a running direction of the belt 132. By using measurement results from the pressure detection points, the load distribution sensor 150 can detect the magnitude and distribution of a vertical load applied from the sole SL of the trainee 900. The load distribution sensor 150 can thereby detect, through the belt 132, the position of the sole SL of the trainee 900 in the standing state and the load applied from the sole SL of the trainee 900. The position of the sole SL is also called a standing position or a treading position of the trainee 900.

The load distribution sensor 150 is connected to the gait state measurement device 100. The gait state measurement device 100 acquires load distribution information as measurement information from the load distribution sensor 150 and measures the gait state of the trainee 900 based on the load distribution information. The gait state is estimated based on the position of the center of gravity, for example, and is also called a gait state index. The gait state measurement device 100 is connected to the system control unit 200 via a wire or wirelessly, and outputs the measured gait state to the system control unit 200.

The system control unit 200 controls various drive units based on the gait state acquired from the gait state measurement device 100. For example, the system control unit 200 is connected, via a wire or wirelessly, to a treadmill drive unit 211, a tension drive unit 214, a harness drive unit 215, and the assistance control unit 220 of the gait assistive device 120. The system control unit 200 drives the treadmill drive unit 211, the tension drive unit 214, and the harness drive unit 215 and sends a control signal to the assistance control unit 220.

The treadmill drive unit 211 includes the aforementioned motor that rotates the belt 132 of the treadmill 131 and a drive circuit for the motor. The system control unit 200 executes rotation control of the belt 132 by sending a drive signal to the treadmill drive unit 211. The system control unit 200 adjusts the rotation speed of the belt 132 according to, for example, a walking speed set by the operator 910. Or the system control unit 200 adjusts the rotation speed of the belt 132 according to the gait state acquired from the gait state measurement device 100.

The tension drive unit 214 includes a motor for applying tension to the front-side wire 134 and a drive circuit for this motor that are provided in the front-side tension unit 135, and a motor for applying tension to the rear-side wire 136 and a drive circuit for this motor that are provided in the rear-side tension unit 137. The system control unit 200 controls each of winding of the front-side wire 134 and winding of the rear-side wire 136 by sending a drive signal to the tension drive unit 214. The system control unit 200 controls a tensile force of each wire by controlling not only a winding action but also drive torque of the motor. Further, the system control unit 200 identifies the timing when the affected leg shifts from a stance leg to a swing leg based on, for example, the gait state of the trainee 900 output from the gait state measurement device 100, and assists the movement of the affected leg by increasing or decreasing the tensile force of each wire in synchronization with that timing.

The harness drive unit 215 includes a motor for applying tension to the harness wire 111 and a drive circuit for this motor that are provided in the harness tension unit 112. The system control unit 200 controls winding of the harness wire 111 and a tensile force of the harness wire 111 by sending a drive signal to the harness drive unit 215. When the system control unit 200 predicts a fall of the trainee 900, for example, it prevents the trainee from falling by winding a certain amount of the harness wire 111.

The assistance control unit 220 is, for example, a microprocessor unit (MPU), and executes control of the gait assistive device 120 by executing a control program provided from the system control unit 200. The assistance control unit 220 notifies the system control unit 200 of a state of the gait assistive device 120. The assistance control unit 220 executes control of start, stop, etc. of the gait assistive device 120 upon receiving a command from the system control unit 200.

The assistance control unit 220 sends a drive signal to a joint drive unit including the motor of the control unit 121 and a drive circuit for the motor, and thereby urges the upper leg frame 122 and the lower leg frames 123 so as to open or close relatively around the hinge shaft Ha. These opening and closing motions assist stretching motion and bending motion of the knee as well as prevent knee fracture. The assistance control unit 220 receives a detection signal from an angle sensor (not shown) that detects the angle formed between the upper leg frame 122 and the lower leg frames 123 around the hinge shaft Ha and calculates an opening angle of the knee joint.

Here, to secure the measurement accuracy of the gait state measurement device 100 and safely perform gait training under control by the system control unit 200, it is necessary for the trainee 900 to tread in a proper area corresponding to the installation area of the load distribution sensor 150. This is because, if the position of the sole SL deviates from the installation area of the load distribution sensor 150, a correct load cannot be obtained by the load distribution sensor 150. Further, in this case, the treading position is located at a left or right end portion of the belt 132, which increases the likelihood of a fall of the trainee 900. The measurement accuracy of the load distribution sensor 150 is sometimes lower at the end portions than at the center, and therefore it is desirable that the trainee 900 avoid treading on the end portions of the load distribution sensor 150.

In Embodiment 1, the walking surface W formed on the belt 132 includes a plurality of target areas that is defined according to the installation position of the load distribution sensor 150. For example, the target areas include a first target area TA1, a second target area TA2, and a third target area TA3.

The first target area TA1 is a predetermined area located inside the installation area of the load distribution sensor 150. The second target area TA2 is an area that encompasses the first target area TA1 and is larger than the first target area TA1. In Embodiment 1, the second target area TA2 coincides with the installation area of the load distribution sensor 150. However, the second target area TA2 may instead be an area that is encompassed by the installation area of the load distribution sensor 150 and smaller than the installation area. The third target area TA3 is an area that encompasses the second target area TA2 and is larger than the second target area TA2. In Embodiment 1, the third target area TA3 coincides with the walking surface W on which the trainee 900 can walk. In the drawing, the entire area of the sole SL of the right leg of the trainee 900 is located inside the first target area TA1.

The shapes of the first target area TA1, the second target area TA2, and the third target area TA3 are all rectangles in Embodiment 1, but are not limited thereto. For example, the shape of the first target area TA1 may be a square or a circle. Since the trainee 900 treads toward the front side in the walking direction, a shape of which the area increases toward the front side in the walking direction is preferable. Accordingly, the shape of the first target area TA1 may be a quadrangle of which the length in the left-right direction increases toward the front side in the walking direction. More specifically, the shape of the first target area TA1 may be a trapezoid of which the longer side of the two opposite parallel sides is located on the front side in the walking direction. The shapes of the second target area TA2 and the third target area TA3 may be defined based on the shapes of the load distribution sensor 150 and the walking surface W, respectively.

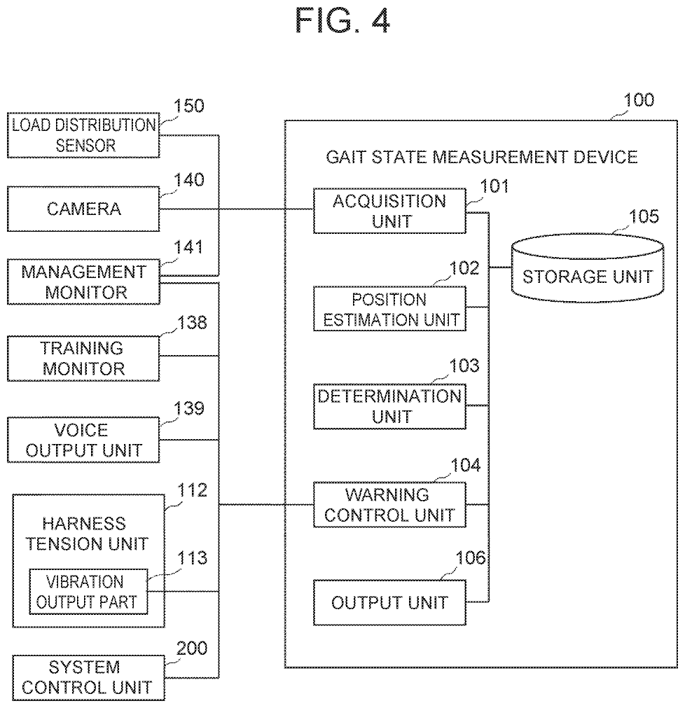

FIG. 4 is a block diagram showing the schematic configuration of the gait state measurement device 100 according to Embodiment 1. The gait state measurement device 100 includes an acquisition unit 101, a position estimation unit 102, a determination unit 103, a warning control unit 104, a storage unit 105, and an output unit 106. These constituent elements of the gait state measurement device 100 are connected to one another.

The acquisition unit 101 acquires load distribution information of the load distribution sensor 150. For example, the acquisition unit 101 is connected to the load distribution sensor 150 and acquires the load distribution information from the load distribution sensor 150. The acquisition unit 101 is connected to the camera 140 and acquires a captured image of the trainee 900 from the camera 140. The captured image of the trainee 900 is specifically an image created by imaging the trainee 900 in walking on the treadmill 131. The acquisition unit 101 is connected to the touch panel 142 of the management monitor 141 and acquires various pieces of information input from the touch panel 142. These various pieces of information include, for example, identification information on the trainee 900 and attribute information on the trainee 900.

The acquisition unit 101 supplies the load distribution information acquired from the load distribution sensor 150 and the captured image acquired from the trainee 900 to the position estimation unit 102. The acquisition unit 101 supplies the various pieces of information acquired from the touch panel 142 to the position estimation unit 102.

The position estimation unit 102 estimates the position of a ground contact area of the sole SL of the trainee 900 based on the load distribution information of the load distribution sensor 150 and the captured image of the trainee 900. Here, the estimated ground contact area of the sole SL will be referred to as a sole area. The position estimation unit 102 supplies information including the estimated position, shape, and area of the sole area (sole area information) to the determination unit 103.

The position estimation unit 102 may calculate the position of the center of gravity based on the load distribution information of the load distribution sensor 150 and calculate the gait state index based on the position of the center of gravity. The position estimation unit 102 may supply the calculated gait state index to the output unit 106 or store it in the storage unit 105. In this case, the position estimation unit 102 may supply the gait state index to the output unit 106, or store it in the storage unit 105, in such a manner that the gait state index is connected to the identification information on the trainee 900 or the attribute information on the trainee 900.

The determination unit 103 estimates which one of a plurality of sole position states into which positions of the sole area are classified applies, based on each of the target areas and the sole area information on the trainee 900. Then, based on the sole position state, the determination unit 103 determines whether to perform a warning to the trainee 900 or the operator 910, and when performing a warning, determines which one of different types of warnings to perform. Then, the determination unit 103 supplies the determination result to the warning control unit 104.

The warning control unit 104 controls other elements of the gait training system 1 so as to perform a warning according to the determination result. That is, the warning control unit 104 controls other elements so as to perform a different type of warning depending on the relationship between each of the target areas and the position of the sole SL of the trainee 900. For example, the warning control unit 104 is communicably connected to the training monitor 138 and the management monitor 141, and displays a warning that indicates, to the trainee 900 and the operator 910, that the treading position of the trainee 900 is not proper. The warning control unit 104 is communicably connected to the voice output unit 139 and outputs the warning to the trainee 900 by voice. The warning control unit 104 is communicably connected to a vibration output part 113 of the harness tension unit 112 and causes the vibration output part 113 to vibrate the harness belt 111a. The warning control unit 104 may cause the vibration output part 113 to vibrate the harness belt 111a through the system control unit 200. In this case, the system control unit 200 is communicably connected to the vibration output part 113. The warning control unit 104 is communicably connected to the system control unit 200, and causes the system control unit 200 to send a drive signal to the treadmill drive unit 211 for reducing the rotation speed of the pulleys 151 of the treadmill 131 or stopping the rotation thereof.

The storage unit 105 is a storage medium that stores information needed for processes in the gait state measurement device 100 or generated information.

The output unit 106 outputs the gait state index calculated by the position estimation unit 102 to the system control unit 200. The output unit 106 may output the gait state index to the training monitor 138 and the management monitor 141. In this case, the output unit 106 may output the gait state index calculated by the position estimation unit 102 in such a manner that the gait state index is connected to the identification information on the trainee 900 or the attribute information on the trainee 900.

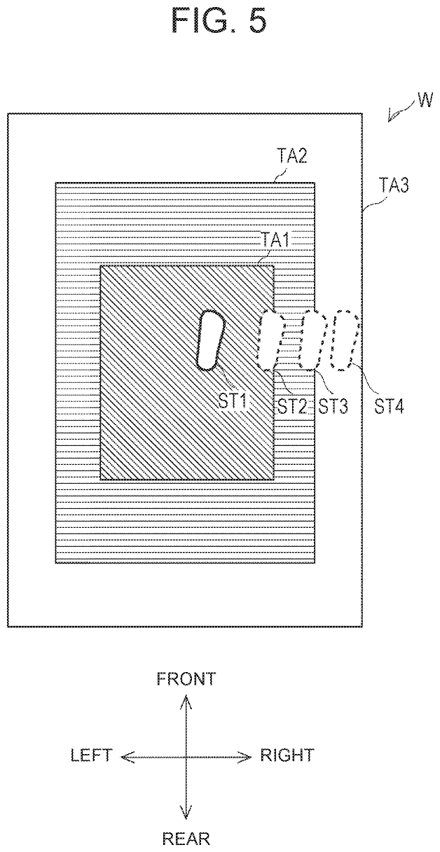

FIG. 5 is a view illustrating the types of warnings according to Embodiment 1. In FIG. 5, sole position states ST1 to ST4 on the walking surface W are shown.

The sole position state ST1 represents the position of the sole area of the trainee 900 when the entire sole area is located inside the first target area TA1. When the position of the sole area is the sole position state ST1, the load distribution sensor 150 can appropriately measure the load, so that the gait state measurement device 100 can appropriately measure the gait state. In this case, the warning control unit 104 does not give a warning.

The sole position state ST2 represents the position of the sole area of the trainee 900 when at least part of the sole area has deviated from the first target area TA1 while the entire sole area of the trainee 900 is located inside the second target area TA2. For example, when the trainee 900 starts to approach an end portion (one of front, rear, left, and right end portions) of a normal practice area, the sole position state ST2 is detected by the determination unit 103. In this case, the load measurement accuracy of the load distribution sensor 150 may decrease at the present or in the future. As a result, the gait state measurement device 100 may become unable to appropriately measure the gait state or unable to measure the gait state. In Embodiment 1, in this case, the warning control unit 104 performs a first warning to avoid this situation. In the first warning, for example, the warning control unit 104 causes the training monitor 138 to display a screen that prompts correction of the treading position, and causes the vibration output part 113 of the harness tension unit 112 to output first vibration. Thus, the trainee 900 can be made aware of the need to return the treading position to the proper position.

The sole position state ST3 represents the position of the sole area of the trainee 900 in the case where at least part of the sole area has deviated from the second target area TA2 while the entire sole area is located inside the third target area TA3. When the sole position state ST3 is detected, the load measurement accuracy of the load distribution sensor 150 decreases. This results in a situation where the gait state measurement device 100 cannot appropriately measure the gait state. In Embodiment 1, in this case, the warning control unit 104 performs a second warning to avoid this situation. In the second warning, for example, the warning control unit 104 causes the training monitor 138 to display a screen that indicates a deviation abnormality, and causes the vibration output part 113 of the harness tension unit 112 to output second vibration that is stronger than the first vibration. In addition, the warning control unit 104 causes the voice output unit 139 to output an alarm sound.

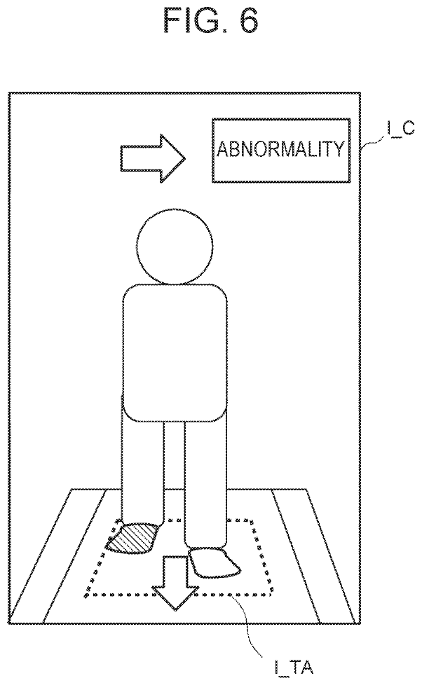

One example of the screen indicating a deviation abnormality in the sole position state ST3 is shown in FIG. 6. FIG. 6 is a view showing one example of the display of the training monitor 138 according to Embodiment 1. An image I_TA indicated by the broken line in FIG. 6 is an image showing the position of the first target area TA1. The training monitor 138 displays an image in which the image I_TA is superimposed on a captured image I_C of the trainee 900. The training monitor 138 may display a captured image I_C on which, in addition to the image I_TA, a comment indicating a deviation abnormality, an arrow indicating a correction direction, and an arrow indicating the front side in the walking direction are further superimposed. The training monitor 138 may display the foot that has deviated in an exaggerated manner as shown in FIG. 6. Such display can effectively prompt the trainee 900 to improve the position.

Turning back to FIG. 5, description will be continued. The sole position state ST4 represents the position of the sole area of the trainee 900 when the entire sole area has deviated from the second target area TA2. The sole position state ST4 is detected, for example, when a state where the load distribution information shows an incorrect value or zero arises suddenly or from the sole position state ST3, and it is recognized from the captured image of the trainee 900 that the sole SL has deviated from the second target area TA2 to an outside. When the sole position state ST4 is detected, this means a situation where not only is the gait state measurement device 100 no longer able to measure the gait state but also it is difficult to perform training safely. In Embodiment 1, in this case, the warning control unit 104 performs a third warning to avoid this situation. In the third warning, for example, the warning control unit 104 causes the training monitor 138 and the management monitor 141 to display a screen that indicates an abnormality, causes the voice output unit 139 to output an alarm sound, and causes the vibration output part 113 of the harness tension unit 112 to output third vibration that is stronger than the second vibration. Thus, the trainee 900 can recognize that the treading position has deviated significantly and become more aware of the need to improve the position. Meanwhile, the operator 910 can accurately grasp the gait state of the trainee 900 and be quickly prompted to make a decision such as whether assistance is needed or whether to suspend the training.

Thus, the gait state measurement device 100 gives warnings in stages using various notification means according to the sole position state, and can thereby allow the trainee 900 or the operator 910 to clearly recognize to what extent the treading position is at the proper position.

As one example, the right leg of the trainee 900 is the affected leg, and only the position of the sole area of the right leg is shown in FIG. 5. However, also for a sole area of the left leg that is a sound leg, sole position states may be similarly defined and the warning control unit 104 may perform similar warnings.

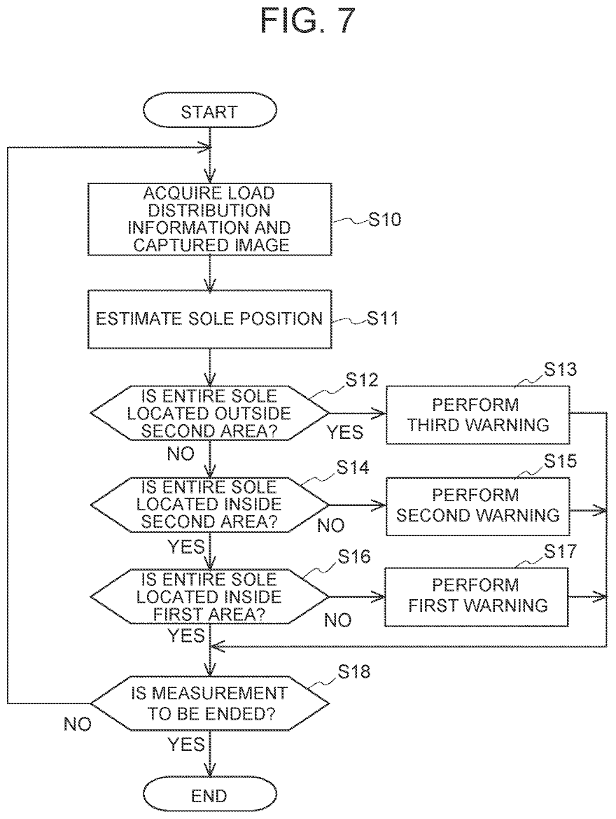

FIG. 7 is a flowchart showing the procedure of a process executed by the gait state measurement device 100 according to Embodiment 1. First, the acquisition unit 101 of the gait state measurement device 100 acquires the load distribution information and the captured image of the trainee 900 (step S10). Next, the position estimation unit 102 estimates the position of the sole area based on the load distribution information and the captured image (step S11). For example, the position estimation unit 102 estimates the position of the sole area, with the sole area being defined as an area where a load equal to or higher than a predetermined threshold value is detected. For example, the position estimation unit 102 detects the leg or the sole of the trainee 900 from the captured image and estimates the position of the sole area based on the detected position. The position estimation unit 102 supplies information on the position of the sole area to the determination unit 103. In this case, the position estimation unit 102 may calculate the gait state index based on the position of the sole area and output the gait state index to the system control unit 200 through the output unit 106.

Next, the determination unit 103 determines whether the entire sole area is located outside the second target area TA2, i.e., whether the sole position state ST4 applies (step S12). When the entire sole area is located outside the second target area TA2, i.e., the sole position state ST4 applies (YES in step S12), the warning control unit 104 performs the third warning (step S13). Then, the warning control unit 104 moves the process to step S18. When the sole position state ST4 does not apply (NO in step S12), the determination unit 103 determines whether the entire sole area is located inside the second target area TA2, i.e., whether the sole position state ST3 does not apply (step S14). When the sole position state ST3 applies (NO in step S14), the warning control unit 104 performs the second warning (step S15). Then, the warning control unit 104 moves the process to step S18. When the entire sole area is located inside the second target area TA2, i.e., when the sole position state ST3 does not apply (YES in step S14), the determination unit 103 moves the process to step S16. In step S16, the determination unit 103 determines whether the entire sole area is located inside the first target area TA1, i.e., whether the sole position state ST1 applies or the sole position state ST2 applies. When the sole position state ST2 applies (NO in step S16), the warning control unit 104 performs the first warning (step S17). Then, the warning control unit 104 moves the process to step S18. When the sole position state ST1 applies (YES in step S16), the warning control unit 104 moves the process to step S18.

In step S18, the warning control unit 104 determines whether to end the measurement. Examples of cases where measurement is ended include a case where training by the gait training system 1 is stopped and a case where the measurement process of the gait state measurement device 100 is stopped by operation of the operator 910. When the measurement is not to be ended (NO in step S18), the warning control unit 104 returns the process to step S10, and otherwise (YES in step S18) the warning control unit 104 ends the process.

The determination unit 103 may predict the future position of the sole area based on transition of the load distribution. In this case, the determination unit 103 may determine the sole position state based on a prediction result in addition to or instead of the current sole position state.

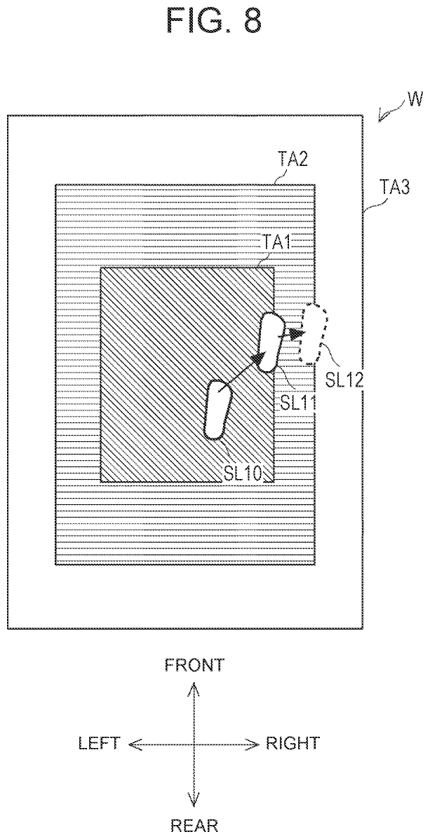

FIG. 8 is a view illustrating a process of predicting the position of the sole area according to Embodiment 1.

The determination unit 103 acquires an estimation history of the position of the sole area of the affected leg corresponding to a predetermined number of times of estimation from the present time. For example, it is assumed that the position of the sole area has moved significantly from SL10 (corresponding to the sole position state ST1) to SL11 (corresponding to the sole position state ST2). In this case, the determination unit 103 predicts that the position of the sole area will be SL12 (corresponding to the sole position state ST3) in the next measurement. In this case, therefore, the determination unit 103 may determine the sole position state as a sole position state ST2′ that is different from the sole position state ST2. In the sole position state ST2′, the warning control unit 104 performs a 1'st warning that is different from the first warning. For example, in the 1'st warning, the warning control unit 104 may cause the vibration output part 113 of the harness tension unit 112 to output vibration that is stronger than the first vibration and weaker than the second vibration. Instead of this, the determination unit 103 may determine the sole position state as the sole position state ST3 in advance. In the sole position state ST3, the warning control unit 104 performs the second warning described above.

Prediction of the future position of the sole area may be performed based on the load distribution inside the sole area, in addition to the estimation history of the position of the sole area. For example, in the case where the position of the sole area is SL11, walking of the trainee 900 is likely to be unstable when a value of a load applied from the sole of the right leg is larger than a value of a load applied from the sole of the left leg by a predetermined threshold value or larger, or when a change over time in a differential value between the left and right load values is equal to or larger than a predetermined threshold value. In this case, therefore, the determination unit 103 may predict that the position of the sole area will be SL12, or the sole position state will be the sole position state ST3, in the next measurement.

Prediction of the future position of the sole area may be performed based on a captured image created by imaging the trainee 900 in walking, in addition to or instead of the load distribution inside the sole area. For example, the determination unit 103 detects the posture of the trainee 900 from the captured image. When it is estimated that the walking is unstable based on the posture, the determination unit 103 may predict that the position of the sole area will be SL12 in the next measurement or that the sole position state will be the sole position state ST3 in the next measurement.

Thus, according to Embodiment 1, the gait state measurement device 100 of the gait training system 1 performs different warnings during gait measurement depending on the positional relationship between the sole of the subject (trainee) and the load distribution sensor 150. Therefore, the subject or the assistant (operator) can be appropriately notified of to what extent the treading position of the subject is at the proper position and of deviation from the proper position. Thus, the subject can be made aware of the need to return the treading position to the proper position. Meanwhile, the assistant can accurately grasp the gait state of the subject and quickly make a decision such as whether assistance is needed.

### Embodiment 2

Next, Embodiment 2 of the disclosure will be described.

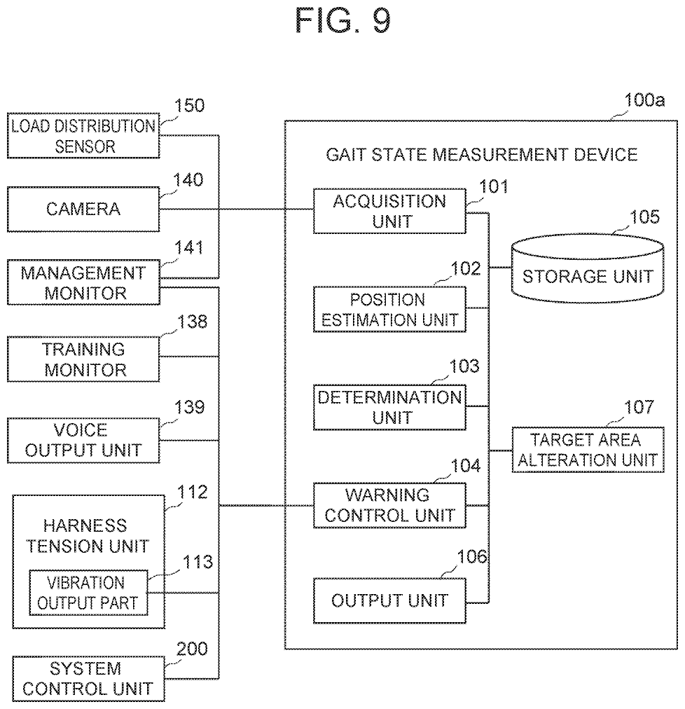

FIG. 9 is a block diagram showing the schematic configuration of a gait state measurement device 100a according to Embodiment 2. In addition to the components and functions of the gait state measurement device 100 according to Embodiment 1, the gait state measurement device 100a according to Embodiment 2 includes a target area alteration unit 107.

The target area alteration unit 107 sets an initial target area based on the attribute information on the trainee 900 or alters the target area from the initial setting based on the attribute information on the trainee 900. Setting or altering the target area means setting or altering at least one of the position, the area, and the shape of the target area. The attribute information is at least one of information showing which of the left and right legs is the affected leg, the stage of rehab, the length of the leg, the size of the foot, the gender, and the age. Based on the treading position of the trainee 900 at the start of training (the initial position of the sole area), the target area alteration unit 107 alters the target area from the initial setting during training. The target area alteration unit 107 may estimate a walking pattern of the trainee 900 from an estimation history of the position of the sole area in past training or from the positions of the sole area estimated during currently proceeding training that correspond to a predetermined number of times of estimation, and may alter the target area based on the walking pattern. The target area may be altered before the start of training or during training.

In Embodiment 2, the target area that is set and altered by the target area alteration unit 107 is the first target area TA1. However, the disclosure is not limited thereto, and the second target area TA2 or the third target area TA3 may also be set and altered by the target area alteration unit 107.

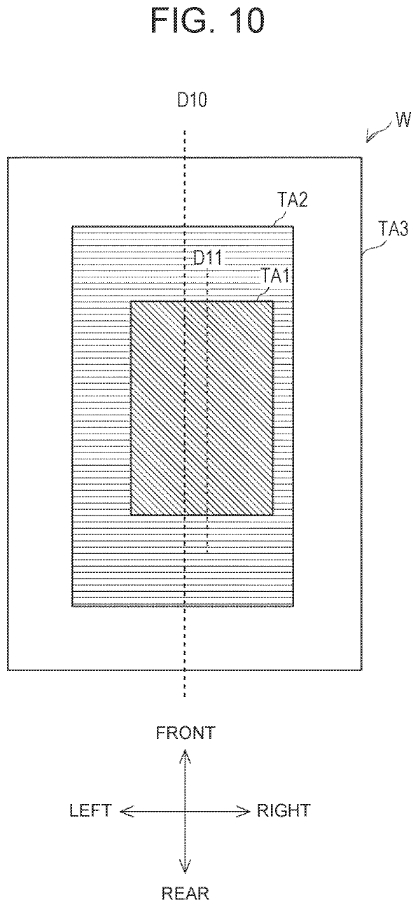

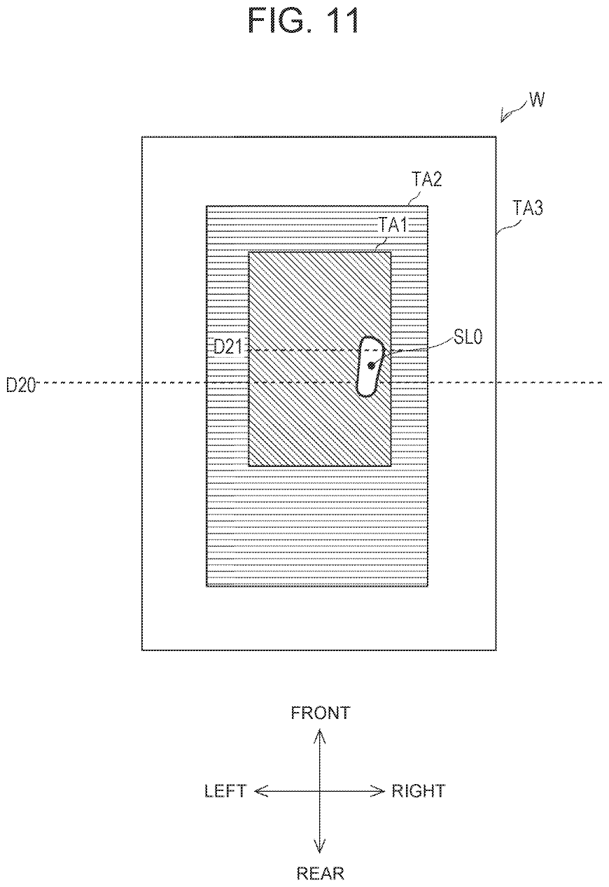

FIG. 10 and FIG. 11 are views showing one example of the target area according to Embodiment 2.

FIG. 10 shows one example of the target areas in the case where the affected leg of the trainee 900 is the left leg. In this case, to attach importance to gait training on the affected leg side, the target area alteration unit 107 sets the first target area TA1 such that a right side (sound leg side) of the first target area TA1 becomes larger and that a left side (affected leg side) thereof becomes smaller. For example, a central axis D11 of the first target area TA1 in the left-right direction is set so as to be shifted toward the right side by a predetermined amount from a central axis D10 of the second target area TA2 in the left-right direction.

FIG. 11 shows one example of the target areas in the case where an initial position SL0 of the sole of the trainee 900 is located on the front side of a central axis D2 of the second target area TA2 in the front-rear direction. In this case, the target area alteration unit 107 sets the first target area TA1 such that the front side of the first target area TA1 becomes larger and that a rear side thereof becomes smaller. Thus, a central axis D21 of the first target area TA1 in the front-rear direction is set so as to be shifted toward the front side by a predetermined amount from a central axis D20 of the second target area TA2 in the front-rear direction. The initial position SL0 of the sole may be the position of the center of gravity of the sole area at the time of treading in an initial stage of measurement.

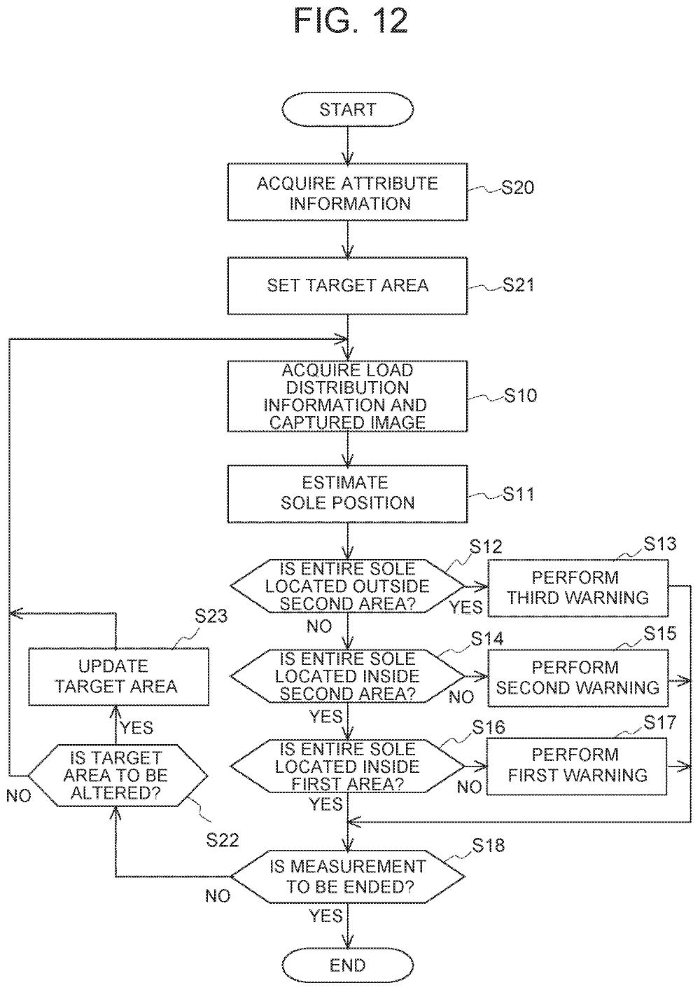

FIG. 12 is a flowchart showing the procedure of a process executed by the gait state measurement device 100a according to Embodiment 2. In addition to steps S10 to S18 shown in FIG. 7, steps shown in FIG. 12 include steps S20 to S23. Description of those steps that are the same as in FIG. 7 will be omitted.

First, the acquisition unit 101 of the gait state measurement device 100a acquires the attribute information on the trainee 900 (step S20). Then, the acquisition unit 101 supplies the attribute information to the target area alteration unit 107. The target area alteration unit 107 sets the target area based on the attribute information (step S21). Then, the gait state measurement device 100a performs the process shown in steps S10 to S18.

Thereafter, when the measurement is to be continued (NO in step S18), the target area alteration unit 107 of the gait state measurement device 100a determines whether to alter the target area (step S22). The target area alteration unit 107 may determine to alter the target area when, for example, the current process is the first cycle. Or the target area alteration unit 107 may determine to alter the target area when a number of cycles required to estimate the walking pattern is reached. When the target area is to be altered (YES in step S22), the target area alteration unit 107 updates the target area (step S23) and returns the process to step S10. On the other hand, when the target area is not to be altered (NO in step S22), the target area alteration unit 107 directly returns the process to step S10.

Thus, according to Embodiment 2, the gait state measurement device 100a automatically changes the target area before measurement or during measurement according to the attribute of the subject (trainee) or the initial position of the sole or the past position of the sole. Thus, the gait state measurement device 100a has enhanced convenience and can perform smooth measurement.

### Embodiment 3

Next, Embodiment 3 of the disclosure will be described. Embodiment 3 is characterized in that the gait state measurement device selects the type of warning and alters the target area based on past and current warnings.

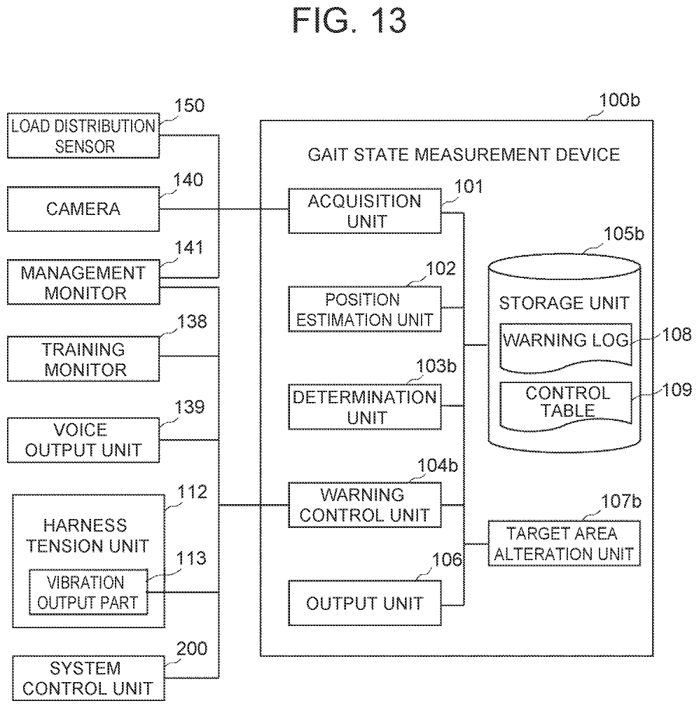

FIG. 13 is a block diagram showing the schematic configuration of a gait state measurement device 100b according to Embodiment 3. The gait state measurement device 100b according to Embodiment 3 has basically the same components and function as the gait state measurement device 100a according to Embodiment 2. However, the gait state measurement device 100b is different in that it includes a determination unit 103b, a warning control unit 104b, a storage unit 105b, and a target area alteration unit 107b instead of the determination unit 103, the warning control unit 104, the storage unit 105, and the target area alteration unit 107.

While the determination unit 103b has basically the same functions as the determination unit 103, it functions also as a record unit that records a history of warnings that are determination results in a warning log 108 to be described later. Based on at least either the number of times of warnings or the pattern of warnings recorded in the warning log 108, the determination unit 103b corrects the type of warning planned to be executed next. For example, when the entire sole area of the trainee 900 is not located inside the first target area TA1, the determination unit 103b first determines to perform the first warning and records this determination in the warning log 108. However, when this case is a case where the first warning has been performed before and the entire sole area of the trainee 900 is not located inside the first target area TA1 within a predetermined time from that first warning, the determination unit 103b corrects the determination result from the first warning to a fourth warning. To correct the determination, the determination unit 103b may use a control table 109 to be described later. Then, the determination unit 103b supplies the corrected determination result to the warning control unit 104b.

In addition to fulfilling the function of the warning control unit 104, the warning control unit 104b controls other elements of the gait training system 1 such that, when the determination result is corrected, a warning according to the corrected determination result is performed. For example, when a corrected determination result to the effect that the fourth warning is to be performed is acquired from the determination unit 103b, the warning control unit 104b causes the management monitor 141 to display a screen that indicates a reminder that the treading position has not improved, and prompts the operator 910 to instruct the trainee 900 to correct the position.

In addition to fulfilling the function of the storage unit 105, the storage unit 105b stores the warning log 108 and the control table 109.

In addition to fulfilling the function of the target area alteration unit 107, the target area alteration unit 107b alters the target area according to at least either the number of times of warnings recorded in the warning log 108 or the pattern of warning. Altering the target area may be altering at least one of the position, the area, and the shape of the target area. To alter the target area, the target area alteration unit 107b may use the control table 109 to be described later.

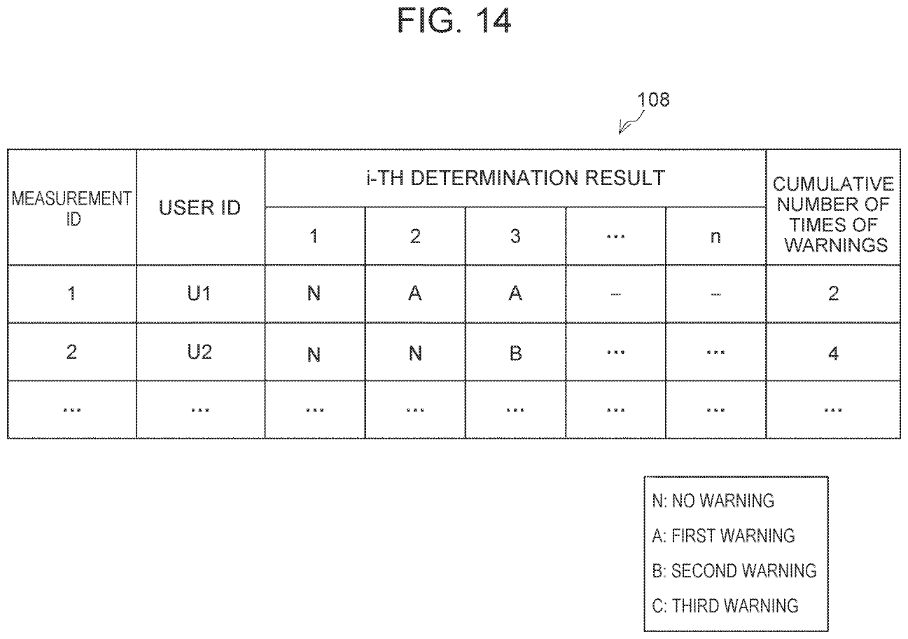

FIG. 14 is a view showing one example of the data structure of the warning log 108 according to Embodiment 3. In the warning log 108, for each measurement identification number (ID), a user ID, history information on determination results, and the cumulative number of times of warnings are connected to one another.

The measurement ID is a number that identifies a series of measurements performed by the gait state measurement device 100b.

The user ID is an ID of the trainee 900.

As the history information on determination results, the first to n-th (n is a natural number not less than two) determination results are stored in chronological order. In the case where a determination result has been corrected, the determination result here may be the determination result before the correction. The determination result may be one of “N” meaning no warning, “A” meaning the first warning, “B” meaning the second warning, and “C” meaning the third warning.

The cumulative number of times of warnings is the number of times of warnings included in the history information on determination results for one measurement.

For example, the history information on determination results for the measurement ID “1” is “N→A→A” and the cumulative number of warnings is two.

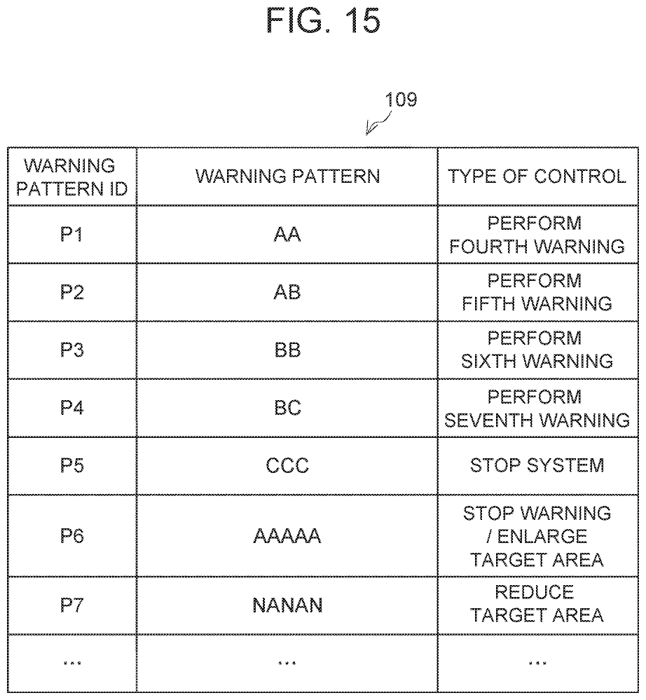

FIG. 15 is a view showing one example of the data structure of the control table 109 according to Embodiment 3. In the control table 109, for each warning pattern ID, a warning pattern and a type of control are connected to each other.

The warning pattern is a chronological pattern of determination results corresponding to a predetermined number of times of determinations up to the present time.

The type of control shows a form of warning to be executed next, a form of alteration of the target area to be executed next, or other forms of control to be executed next.

For example, the warning pattern ID “P1” shows that, if the first warning is performed twice consecutively (A→A), the determination unit 103b corrects the determination result to the fourth warning. The warning pattern ID “P2” shows that, if the second warning is performed directly after the first warning (A→B), the determination unit 103b will correct the determination result to a fifth warning.

If, as in the warning pattern ID “P6,” the first warning is performed a predetermined number of times or a larger number of times consecutively (in FIG. 15, A→A→A→A→A), such control that allows smooth proceeding of measurement is performed. This is because, even when the sole has deviated from the first target area TA1 that is a normal training area, if that state continues stably for a predetermined time, it is highly likely that there is no problem in terms of measurement accuracy as well as safety. Specifically, the determination unit 103b corrects the determination result so as to stop warnings, and the target area alteration unit 107b enlarges the target area by a predetermined ratio.

Depending on the purpose of measurement, the warning pattern ID “P6” may be considered to pose a problem in executing measurement. In this case, the item of the type of control may be changed. For example, the determination unit 103b may correct the determination result so as to execute a stronger warning, and the target area alteration unit 107b may reduce the target area by a predetermined ratio.

For example, in the warning pattern ID “P5,” if the third warning is performed a predetermined number of times or a larger number of times consecutively (in FIG. 15, C→C→C), it is determined that it is dangerous to continue measurement, and control is performed to suspend the measurement. Specifically, the determination unit 103b corrects the determination result to a determination result to the effect that control of the system control unit 200 is to be stopped.

Thus, according to Embodiment 3, the gait state measurement device 100b selects the type of warning to be executed next and alters the target area to be used next based on the past and current determination results, which can enhance the convenience according to the actual situation of measurement.

While the disclosure has been described as a hardware configuration in the above embodiments, the disclosure is not limited thereto. The various processes involved in the gait state measurement method of the disclosure can also be realized by causing a processor of a computer to execute computer programs.

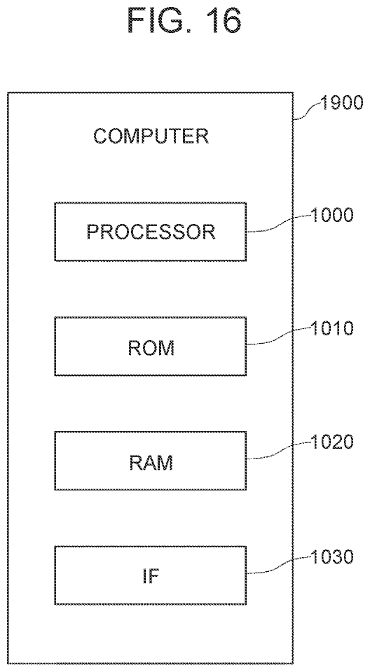

FIG. 16 is a schematic configuration diagram of a computer 1900 that is used as the gait state measurement devices according to Embodiments 1 to 3.

The computer 1900 has a processor 1000, a read-only memory (ROM) 1010, a random-access memory (RAM) 1020, and an interface (IF) 1030 as major hardware components. The processor 1000, the ROM 1010, the RAM 1020, and the interface 1030 are connected to one another through a data bus or the like.

The processor 1000 functions as a calculation device that performs a control process, a calculation process, etc. The processor 1000 may be a central processing unit (CPU), a graphics processing unit (GPU), a field-programmable gate array (FPGA), a digital signal processor (DSP), or an application specific integrated circuit (ASIC), or a combination thereof. The ROM 1010 functions to store control programs, calculation programs, etc. executed by the processor 1000. The RAM 1020 functions to temporarily store processing data etc. The interface 1030 allows signals to be input from and output to the outside via a wire or wirelessly. The interface 1030 receives a user's operation of inputting data and displays information to the user. For example, the interface 1030 communicates with the load distribution sensor 150, the camera 140, the management monitor 141, the training monitor 138, the voice output unit 139, the vibration output part 113, and the system control unit 200.

In the above example, the program includes a group of commands (or software codes) to cause a computer to perform one or more of the functions described in the embodiments when loaded to the computer. The program may be stored in a non-transitory computer-readable medium or a tangible storage medium as one example of the ROM 1010. Examples of computer-readable media and tangible storage media include, but are not limited to, a random-access memory (RAM), a read-only memory (ROM), a flash memory, a solid-state drive (SSD), and other memory technologies, a CD-ROM, a digital versatile disc (DVD), a Blu-ray® disc, and other optical disc storages, a magnetic cassette, a magnetic tape, a magnetic disk storage, and other magnetic storage devices. The program may be sent in a transitory computer-readable medium or a communication medium. Examples of transitory computer-readable media and communication media include, but are not limited to, electrical, optical, and acoustic signals, and other forms of propagation signals.

In the above embodiments, the computer 1900 is formed by a computer system including a personal computer, a word processor, and the like. However, without being limited thereto, the computer 1900 can also be formed by a server of a local area network (LAN), a host of computer (personal computer) communication, a computer system connected to the Internet, and the like. It is also possible to distribute functions among devices on a network and configure the computer 1900 by the whole network. Thus, the constituent elements of the gait state measurement device may be distributed among different devices.

The disclosure is not limited to the above embodiments but can be changed as necessary within such a scope that does not depart from the gist of the disclosure. For example, in Embodiment 1, the gait state measurement device 100 performs the third warning when the sole position state is the sole position state ST4 shown in FIG. 5, but the third warning is not essential. Accordingly, in step S10 of FIG. 7, the captured image of the trainee 900 need not be used as the basis for estimating the position of the sole area by the position estimation unit 102.

In each of the above embodiments, the gait state measurement device calculates the gait state index, but instead of the gait state measurement device, the system control unit 200 may calculate the gait state index. In this case, the position estimation unit 102 of the gait state measurement device may send information on the position of the sole area to the system control unit 200 through the output unit 106 upon estimating the position of the sole area.

In each of the above embodiments, the example in which the trainee 900 is a hemiplegia patient suffering from paralysis in one of his or her legs has been described, but the disclosure is not limited thereto. For example, the trainee 900 may be a patient suffering from paralysis in both legs. In this case, the trainee 900 receives training by wearing the gait assistive device 120 on both legs. Or the trainee 900 need not wear the gait assistive device 120 on either leg.

What is claimed is:

1. A gait state measurement system that measures a gait state of a subject who is walking on a walking surface formed on a belt running along a circulation track, the gait state measurement system comprising:
an acquisition unit that acquires measurement information of a load distribution sensor that detects a load of the subject through the belt;
a position estimation unit that estimates a position of a sole of the subject based on the measurement information; and
a warning control unit that performs a different type of warning depending on a relationship between the position of the sole of the subject and each of target areas that are defined according to an installation area of the load distribution sensor.

2. The gait state measurement system according to claim 1, wherein:
the target areas include a first target area that is located inside the installation area and a second target area that encompasses the first target area;
the warning control unit performs a first warning when at least part of the sole of the subject deviates from the first target area while an entire sole of the subject is located inside the second target area; and
the warning control unit performs a second warning when at least part of the sole of the subject deviates from the second target area.

3. The gait state measurement system according to claim 2, wherein:
the position estimation unit estimates the position of the sole of the subject based on a captured image created by imaging the subject in walking and on the measurement information; and
the warning control unit performs a third warning when the entire sole of the subject deviates from the second target area.

4. The gait state measurement system according to claim 2, wherein the warning control unit performs a fourth warning when the entire sole of the subject is not located inside the first target area within a predetermined time from the first warning.

5. The gait state measurement system according to claim 1, comprising a target area alteration unit that alters at least one of a position, an area, and a shape of the target area based on at least one of attribute information on the subject and an initial position of the sole of the subject.

6. The gait state measurement system according to claim 1, comprising:
a record unit that records a history of the warnings in a warning log; and
a target area alteration unit that alters at least one of a position, an area, and a shape of the target area based on at least either the number of times of warnings or a chronological pattern of warnings recorded in the warning log.

7. A gait state measurement method that measures a gait state of a subject who is walking on a walking surface formed on a belt that runs along a circulation track, the gait state measurement method comprising:
an acquisition stage of acquiring measurement information of a load distribution sensor that detects a load of the subject through the belt;
a position estimation stage of estimating a position of a sole of the subject based on the measurement information; and
a warning control stage of performing a different type of warning depending on a relationship between the position of the sole of the subject and each of target areas that are defined according to an installation area of the load distribution sensor.

8. A program that causes a computer to execute a gait state measurement method that measures a gait state of a subject who is walking on a walking surface formed on a belt that runs along a circulation track, wherein the gait state measurement method includes:
an acquisition stage of acquiring measurement information of a load distribution sensor that detects a load of the subject through the belt;
a position estimation stage of estimating a position of a sole of the subject based on the measurement information; and
a warning control stage of performing a different type of warning depending on a relationship between the position of the sole of the subject and each of target areas that are defined according to an installation area of the load distribution sensor.
